(19) United States
(12) Patent Application Publication
(10) Pub. No.: US 2026/0120806 A1
(43) Pub. Date: Apr. 30, 2026

(54) DNA SEQUENCE EMBEDDING DEVICE AND METHOD BASED ON ATTENTION MECHANISM

(71) Applicant: UIF (UNIVERSITY INDUSTRY FOUNDATION), YONSEI UNIVERSITY, Seoul (KR)

(72) Inventors: ALBERT NO, Seoul (KR); KYELIM LEE, Seoul (KR)

(73) Assignee: UIF (UNIVERSITY INDUSTRY FOUNDATION), YONSEI UNIVERSITY, Seoul (KR)

(21) Appl. No.: 18/933,561

(22) Filed: Oct. 31, 2024

(30) Foreign Application Priority Data
Oct. 24, 2024 (KR) 10-2024-0146873

(51) Int. Cl. G16B 40/00; G06N 3/045; G06N 3/0499

(52) U.S. Cl. CPC G16B 40/00; G06N 3/045; G06N 3/0499

(57) ABSTRACT

The present invention relates to a DNA sequence embedding device and method based on an attention mechanism, and the device includes an encoder module that transforms a DNA sequence into an embedding vector, a decoder module that receives the embedding vector and transforms the embedding vector into a sequence vector, a cross-attention module that generates an attention map based on a correlation between the sequence vectors, a matrix transformation module that transforms the DNA sequence into a dynamic program-based matrix, and a learning module that performs learning to minimize a loss between the attention map and the matrix.

### CROSS-REFERENCE TO RELATED APPLICATION

This application claims, under 35 U.S.C. § 119 (a), the benefit of Korean Patent Application No. 10-2024-0146873 filed on Oct. 24, 2024, the entire contents of which are incorporated herein by reference.

### TECHNICAL FIELD

The present invention relates to DNA sequence embedding technology, and more specifically, to a DNA sequence embedding device and method based on an attention mechanism capable of accurately and efficiently approximating an edit distance between two sequences in DNA sequence data to improve DNA sequence embedding performance.

### BACKGROUND

With the development of DNA sequencing technology, a vast amount of DNA data has been accumulated, and analysis of such data has become a very difficult task. Accordingly, interest in data-based bioinformatics research has rapidly increased. In this case, the concept of an edit distance (also called a Levenshtein distance) may be used as an important tool. The edit distance refers to a scheme for measuring the minimum number of editing tasks (insertion, deletion, replacement, and the like) required to transform one string into another string. This distance may be very useful in various bioinformatics tasks such as sequence clustering and multiple sequence alignment (MSA). In particular, sequences are read several times in tasks such as DNA storage, and, when results thereof are clustered, the quality of a reading procedure can be improved. In the field of phylogenetics, DNA, RNA, or protein sequences of different species are compared in order to analyze similarities and differences therebetween, and evolutionary relationships are inferred based on the analysis. In this case, the differences between the sequences may be quantitatively measured through the edit distance, and the similarities may be compared. In other words, hierarchical clustering of data through edit distance calculation is required in order to visualize the evolutionary relationship between the species based on the similarity and the difference between the sequences.

For existing edit distance calculations, a dynamic programming-based algorithm, especially, a Needleman-Wunsch (NW) algorithm, has been used. The NW algorithm is known to be an effective method for calculating an edit distance between two sequences, but computational complexity increases exponentially with a sequence length, making it difficult to apply the NW algorithm to large-scale datasets.

To solve this problem, a scheme for approximating the edit distance by transforming the sequence into a vector space through an embedding network utilizing deep learning has recently been studied. However, such methods generally do not sufficiently reflect the alignment information between the sequences, resulting in limited performance.

### PRIOR ART DOCUMENT

### Patent Document

Korean Patent Publication No. 10-2022-0042300 (Apr. 5, 2022)

### DESCRIPTION

### Problem to be Solved

An embodiment of the present invention is intended to provide a DNA sequence embedding device based on an attention mechanism and method capable of accurately and efficiently approximating an edit distance between two sequences in DNA sequence data based on an attention mechanism to improve the performance of DNA sequence embedding.

Another embodiment of the present invention is intended to provide a DNA sequence embedding device based on an attention mechanism and method capable of improving edit distance preserving performance through a sequence embedding framework that is a combination of an NW matrix and an attention mechanism.

### Solution

In the embodiments, a DNA sequence embedding device based on an attention mechanism includes an encoder module configured to transform a DNA sequence into an embedding vector; a decoder module configured to receive the embedding vector and transform the embedding vector into a sequence vector; a cross-attention module configured to generate an attention map based on a correlation between the sequence vectors; a matrix transformation module configured to transform the DNA sequence into a dynamic program-based matrix; and a learning module configured to perform learning to minimize a loss between the attention map and the matrix.

The encoder module may represent the edit distance between the DNA sequences as a distance in a vector space to generate the embedding vector.

The encoder module may include a neural network that is trained to minimize an edit distance loss function [symbol] _{enc}(θ) so that a difference between an actual edit distance between the DNA sequences and a distance between the embedding vectors is decreased.

The decoder module may transform the embedding vectors so that information close to original sequences can be extracted, thereby allowing the sequence vectors to reflect alignment information between the original sequences.

The decoder module may include a multi-head self-attention (MSA) unit configured to perform first learning based on a plurality of attention heads to learn a correlation between a first element and a second element of the embedding vector; and a feedforward network (FFN) unit configured to perform second learning based on a multilayer perceptron structure to learn a complex relationship through nonlinear transformation of a result of learning the correlation.

The cross-attention module may perform calculation of the correlation and a row-wise Softmax operation based on a query-key-value structure of the sequence vector to generate the attention map probabilistically representing a relationship between the sequence vectors.

The matrix transformation module may implement a dynamic program-based matrix as a Needleman-Wunsch (NW) matrix.

The learning module may measure a probabilistic difference between the attention map and the matrix and perform the learning through a regularization term for minimizing the difference.

In the embodiments, a DNA sequence embedding method based on an attention mechanism performed in a DNA sequence embedding device based on an attention mechanism includes an encoder step of transforming a DNA sequence into an embedding vector; a decoder step of receiving the embedding vector and transforming the embedding vector into a sequence vector; a cross-attention step of generating an attention map based on a correlation between the sequence vectors; a matrix transformation step of transforming the DNA sequence into a dynamic program-based matrix; and a learning step of performing learning to minimize a loss between the attention map and the matrix.

### EFFECT

The disclosed technology can have the following effects. However, since this does not mean that a specific embodiment should include all of the following effects or only the following effects, the scope of the disclosed technology should not be understood as being limited thereby.

With the DNA sequence embedding device and method based on an attention mechanism according to an embodiment of the present invention, it is possible to accurately and efficiently approximate the edit distance between two sequences in DNA sequence data based on an attention mechanism and improve DNA sequence embedding performance.

With the DNA sequence embedding device and method based on an attention mechanism according to an embodiment of the present invention, it is possible to improve edit distance preserving performance through a sequence embedding framework that is a combination of an NW matrix and an attention mechanism.

Therefore, according to the present invention, it is possible to accurately calculate the similarity between the DNA sequences and improve the efficiency of a data analysis task such as clustering.

### BRIEF DESCRIPTION OF THE DRAWINGS

FIG. 1 is a diagram illustrating a DNA sequence embedding device based on an attention mechanism according to the present invention.

FIG. 2 is a flowchart illustrating a DNA sequence embedding method based on an attention mechanism according to the present invention.

FIG. 3 is a diagram illustrating a structure of a decoder module in an NWA framework of FIG. 1.

FIG. 4 is a diagram illustrating a structure of a cross-attention module in the NWA framework of FIG. 1.

FIG. 5 is a diagram illustrating a result of scaling a regularization term weight adjustment parameter λ in a learning model of the NWA framework proposed in the present invention.

FIG. 6 is a diagram illustrating a visualization of an original NW matrix and an attention map learned with NWA.

### DETAILED DESCRIPTION

Specific structural or functional descriptions in the embodiments of the present disclosure introduced in this specification or application are only for description of the embodiments of the present disclosure. The descriptions should not be construed as being limited to the embodiments described in the specification or application. The present disclosure may, however, be embodied in many different forms, but should be construed as covering modifications, equivalents or alternatives falling within ideas and technical scopes of the present disclosure. Further, since effects disclosed herein do not mean that a specific embodiment should include all or only the effects, the scope of the present disclosure should not be construed as being limited thereto.

Meanwhile, the meaning of terms described herein will be understood as follows.

It will be understood that, although the terms “first”, “second”, etc. may be used herein to distinguish one element from another element, these elements should not be limited by these terms. For instance, a first element discussed below could be termed a second element without departing from the teachings of the present disclosure. Similarly, the second element could also be termed the first element.

It will be understood that when an element is referred to as being “coupled” or “connected” to another element, it can be directly coupled or connected to the other element or intervening elements may be present therebetween. In contrast, it should be understood that when an element is referred to as being “directly coupled” or “directly connected” to another element, there are no intervening elements present. Other expressions that explain the relationship between elements, such as “between”, “directly between”, “adjacent to” or “directly adjacent to” should be construed in the same way.

In the present disclosure, the singular forms are intended to include the plural forms as well, unless the context clearly indicates otherwise. It will be further understood that the terms “comprise”, “include”, “have”, etc. when used in this specification, specify the presence of stated features, integers, steps, operations, elements, components, and/or combinations of them but do not preclude the presence or addition of one or more other features, integers, steps, operations, elements, components, and/or combinations thereof.

In each step, reference characters (e.g. a, b, c, etc.) are used for the convenience of description. The reference characters do not designate the order of the steps, and the steps may be performed in a different order unless the context clearly indicates otherwise. That is, the steps may be performed in the specified order, may be performed substantially simultaneously, or may be performed in a reverse order.

The present disclosure can be implemented as a computer-readable code on a computer-readable recording medium. The computer-readable recording medium includes all types of recording devices in which data readable by a computer system is stored. Examples of the computer-readable recording medium include ROM, RAM, CD-ROM, magnetic tape, floppy disk, an optical data storage device, etc. In addition, the computer-readable recording medium may be distributed in a computer system connected via a network, so that computer-readable codes may be stored and executed in a distributed manner.

Unless otherwise defined, all terms including technical and scientific terms used herein have the same meaning as commonly understood by one of ordinary skill in the art to which the present disclosure belongs. It will be further understood that terms used herein should be interpreted as having a meaning that is consistent with their meaning in the context of this specification and the relevant art and will not be interpreted in an idealized or overly formal sense unless expressly so defined herein.

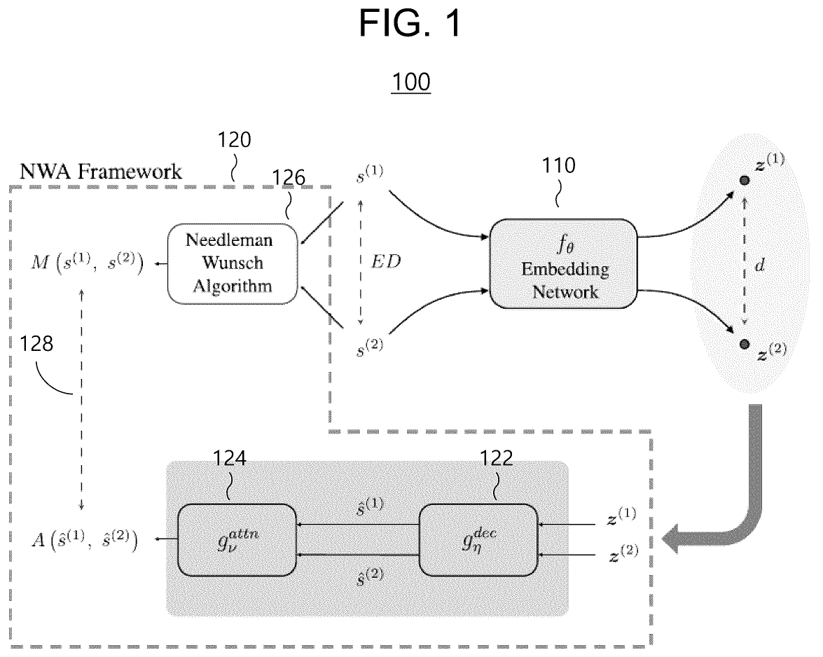

FIG. 1 is a diagram illustrating a DNA sequence embedding device based on an attention mechanism according to the present invention.

Referring to FIG. 1, a DNA sequence embedding device based on an attention mechanism (hereinafter, referred to as a DNA sequence embedding device) 100 may include an encoder module 110 and a Needleman-Wunsch Attention (NWA) framework 120.

The NWA framework 120 is a sequence embedding framework that is a combination of an NW matrix and an attention mechanism. The attention mechanism is a technique for modeling a relationship between sequences by finding out a correlation between elements of the sequence. The NWA framework 120 may learn the alignment information between the sequences and compare the information with the NW matrix to improve the learning performance of an embedding network. To this end, the NWA framework 120 may include a decoder module 122, a cross-attention module 124, a matrix transformation module 126, and a learning module 128.

The encoder module 110 may transform a DNA sequence into an embedding vector. In an embodiment, the encoder module 110 may generate the embedding vector by representing an edit distance between DNA sequences as a distance in a vector space. Through this, the similarity or difference between respective DNA sequences may be efficiently compared and analyzed in the vector space. The DNA sequence generally includes four bases A, T, C, and G. The encoder module 110 may represent each of these bases as a unique vector. For example, the base may be mapped to a unique position in an embedding dimension, and a numeric vector may be assigned to each base. The encoder module 110 may transform an edit distance (insertion, deletion, substitution, or the like) between two sequences into the similarity to represent the edit distance between the sequences as a distance in the vector space, and represent a difference between the sequences as the distance in the vector space. For the encoder module 110, various deep learning models such as a transformer architecture may be used.

In an embodiment, the encoder module 110 may be configured as a neural network that is trained to minimize an edit distance loss function [symbol] _{enc} (θ) so that a difference between an actual edit distance between the DNA sequences and a distance between the embedding vectors is decreased. The edit distance is a quantitative index indicating a difference between two DNA sequences, and means the minimum number of tasks required to transform one sequence into another sequence through a transformation such as insertion, deletion, or substitution. The embedding vector is a method of representing DNA sequences in a high-dimensional vector space, and the similarity between two sequences may be measured by using a method such as an Euclidean distance or a cosine similarity between embedding vectors. The edit distance loss function [symbol] _{enc} (θ) may be used to minimize a difference between an actual edit distance between two sequences and a distance difference between the embedding vectors calculated in the vector space. A loss function [symbol] _{ed} (θ) of the encoder module 110 may be the edit distance loss function [symbol] _{enc} (θ), and is as shown in Formula 1 below.

$${ℒ}_{ed} ( θ ) = {\sum }_{{s}^{( 1 )} , {s}^{( 2 )}} {( ED ( {s}^{( 1 )} , {s}^{( 2 )} ) - d ( {f}_{θ} ( {s}^{( 1 )} ) , {f}_{θ} ( {s}^{( 2 )} ) ) )}^{2} \qquad [ Formula 1 ]$$

Here, the sequence is s=s₁, s₂, . . . , sₙ, f_{θ} is a neural network that transforms the sequence into the embedding vector, and a sum of loss functions is calculated for a given sequence pair s⁽¹⁾, s⁽²⁾ ∈Sⁿ.

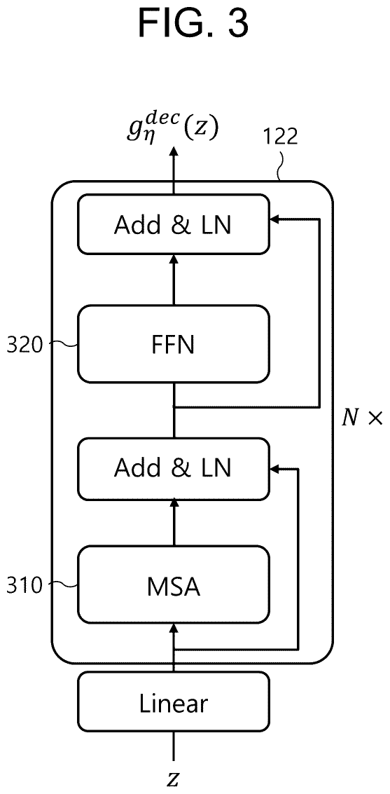

The decoder module 122 may receive embedding vectors z⁽¹⁾ and z⁽²⁾ and transform the embedding vectors z⁽¹⁾ and z⁽²⁾ into sequence vectors ŝ⁽¹⁾ and ŝ⁽²⁾. In an embodiment, the decoder module 122 may transform the embedding vectors so that information close to the original sequences can be extracted, thereby allowing the sequence vectors to reflect alignment information between the original sequences. The sequence vector is a vector required to restore original DNA sequences, and reflects how the bases A, T, C, and G of the sequence are aligned. The decoder module 122 may use the attention mechanism to extract the information close to the original sequence from information included in the embedding vector. The attention mechanism may learn which base of the DNA sequence each part of the embedding vector corresponds to, thereby reflecting the alignment information between the sequences. Alignment information between the DNA sequences has an important biological meaning, and the decoder module 122 may utilize this alignment information to restore a sequence structure similar to the original sequence from the embedding vector. The decoder module 122 may perform first learning based on a plurality of attention heads to learn a correlation between a first element and a second element of the embedding vector, and perform second learning based on a multilayer perceptron structure to learn a complex relationship through nonlinear transformation of a result of learning the correlation. This will be described in detail with reference to FIG. 3.

The cross-attention module 124 may generate an attention map A(ŝ⁽¹⁾,ŝ⁽²⁾)) based on a correlation between the sequence vectors ŝ⁽¹⁾ and ŝ⁽²⁾. The cross-attention module 124 may perform calculation of the correlation and a row-wise Softmax operation based on a query-key-value structure of the sequence vector to generate an attention map probabilistically representing a relationship between the sequence vectors. The cross-attention module 124 may ascertain a relationship between two different sequence vectors and represent the relationship as an attention map. The attention map may quantitatively represent the similarity and correlation between the sequence vectors. Here, the query represents information of one sequence vector (for example, ŝ⁽¹⁾) and is used to evaluate a correlation with the other sequence vector (for example, ŝ⁽²⁾). The key contains information of the other sequence vector (for example, ŝ⁽²⁾) to be compared, and the key of each sequence provides information necessary to evaluate a correlation with a query Q. The value is data containing actual correlation information, and is used to construct a final attention map by being weighted according to a correlation between the query Q and the key K. In an embodiment, the cross-attention module 124 may calculate a dot product between each query and all keys to measure similarity between the two vectors, and scale a result of calculating the dot product to calculate the correlation. Further, the cross-attention module 124 may apply a correlation score calculated for each query to a softmax function for conversion into a probability value, and provide a probability distribution for all the keys for each query through the probability value. This probability distribution is based on a relationship between the query and the key, and may clearly represent a correlation between the two sequences. The cross-attention module 124 may represent the relationship between the sequence vectors as an attention map to visually represent the similarity between the respective sequence.

The matrix transformation module 126 may transform the DNA sequence into a dynamic program-based matrix. The matrix transformation module 126 may implement the dynamic program-based matrix as a Needleman-Wunsch (NW) matrix. The dynamic programming is a methodology of dividing a problem into smaller subproblems to solve an optimization problem. The NW matrix includes information necessary to find optimal alignment between two sequences, and plays an important role in analysis of the similarity between the sequences and various bioinformatics tasks.

The learning module 128 may perform learning to minimize a loss between the attention map and the matrix. The learning module 128 may measure a probabilistic difference between the attention map and the matrix and perform learning through a regularization term for minimizing the difference. In an embodiment, the learning module 128 can measure the probabilistic difference between the attention map and the NW matrix using a distance measurement method, and introduce an additional regularization term to the loss function to minimize the difference between the attention map and the NW matrix during a learning process. That is, the learning module 128 may additionally introduce the regularization term in the learning process so that the alignment information between the sequences is better reflected in an embedding process.

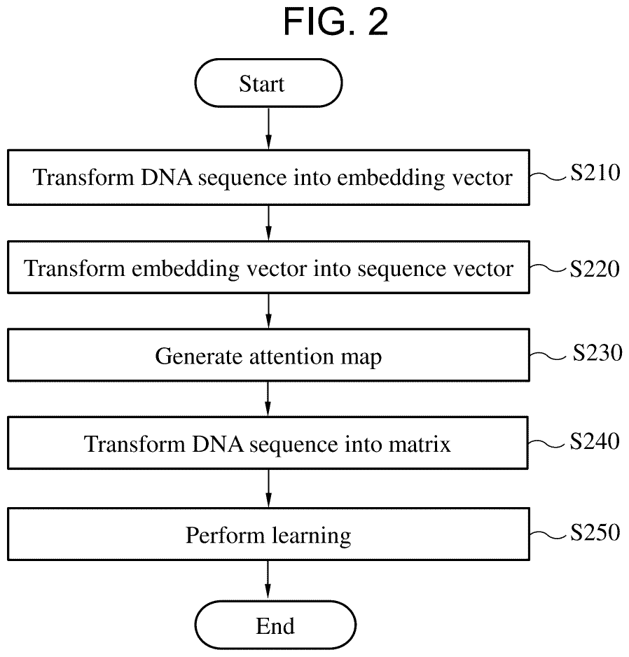

FIG. 2 is a flowchart illustrating a DNA sequence embedding method based on an attention mechanism according to the present invention.

Referring to FIG. 2, the DNA sequence embedding device 100 may transform a DNA sequence into an embedding vector through the encoder module 110 (step S210). The DNA sequence embedding device 100 may receive the embedding vector and transform the embedding vector into the sequence vector through the decoder module 122 (step S220).

Further, the DNA sequence embedding device 100 may generate the attention map based on the correlation between the sequence vectors through the cross-attention module 124 (step S230). The DNA sequence embedding device 100 may transform the DNA sequence into a dynamic program-based matrix through the matrix transformation module 126 (step S240).

Further, the DNA sequence embedding device 100 may perform learning to minimize the loss between the attention map and the matrix through the learning module 128 (step S250).

FIG. 3 is a diagram illustrating a structure of the decoder module in the NWA framework of FIG. 1.

Referring to FIG. 3, the NWA framework 120 may be implemented to learn a sequence alignment structure through the attention map based on similarity between the attention map and the NW matrix. The decoder module 122 may maintain the alignment information between the sequences by transforming an embedding vector z transformed by the encoder module 110 back into the sequence vector ŝ. The decoder module 122 may be structured to be used repeatedly in N (N is a natural number) blocks, the respective blocks may have the same structure, and the relationship between the sequences may be precisely learned as the blocks are repeated.

The decoder module 122 may receive the embedding vector z and linearly transform the embedding vector z to adjust a dimension of the embedding vector z. The decoder module 122 may include a Multi-Head Self-Attention (MSA) unit 310 and a Feedforward Network (FFN) unit 320, similar to a transformer encoder.

The MSA unit 310 may perform the first learning based on a plurality of attention heads to learn the correlation between the first element and the second element of the embedding vector z. The MSA unit 310 may learn the correlation through self-attention as the respective elements of the embedding vector z interact. In this process, the similarity, the association, and the like between the sequences may be learned. The MSA unit 310 may perform normalization by adding a previous input value to an attention result and transfer a normalization result to the FFN unit 320.

The FFN unit 320 may perform the second learning based on a multilayer perceptron structure to learn a complex relationship through nonlinear transformation of a result of learning the correlation.

The decoder module 122 may restore the sequence vector by repeating the first and second learning processes N times.

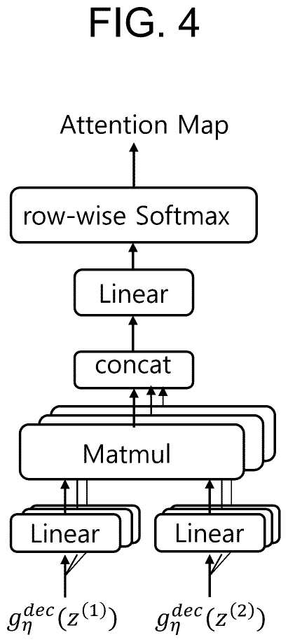

FIG. 4 is a diagram illustrating a structure of the cross-attention module in the NWA framework of FIG. 1.

Referring to FIG. 4, the cross-attention module 124 may generate the attention map, and receive the outputs

$${g}_{η}^{dec} ( {z}^{( 1 )} ) and {g}_{η}^{dec} ( {z}^{( 2 )} )$$

of the decoder module 122 to generate n×n attention maps ŝ⁽¹⁾ and ŝ⁽²⁾. The outputs

$${g}_{η}^{dec} ( {z}^{( 1 )} ) and {g}_{η}^{dec} ( {z}^{( 2 )} )$$

of the decoder module 122 are embedding vectors corresponding to the respective sequences. The cross-attention module 124 may linearly transform the two input sequence vectors to generate query, key, and value vectors for each sequence. The cross-attention module 124 may perform a dot product between the query vector and the key vector to calculate the correlation between the two sequences, and measure whether an element of each sequence is related to which element of the other sequence. The dot product calculation may be performed as calculation of an attention score indicating a correlation between the query and the key. The cross-attention module 124 combines the attention scores obtained through the dot product calculation into a single matrix through a vector concatenation process and then performs linear transformation again. The cross-attention module 124 finally performs a row-wise Softmax operation to convert the attention scores into a probability distribution so that respective rows of an attention matrix is summed into 1. The finally generated attention map is a result of reflecting an interaction between the two sequences, and may represent a relationship between elements of the two sequences. That is, when g_{η,v} is defined as an attention map generation network of [symbol] ^{m}× [symbol] ^{m}→ [symbol] ^{n×m n}, this is a simple combination of the decoder module 122 and the cross-attention module 124, as shown in Formula 2 below.

$${g}_{η , v} ( {z}^{( 1 )} , {z}^{( 2 )} ) = {g}_{v}^{attn} ( {g}_{η}^{dec} ( {z}^{( 1 )} ) , {g}_{η}^{dec} ( {z}^{( 2 )} ) ) \qquad [ Formula 2 ]$$

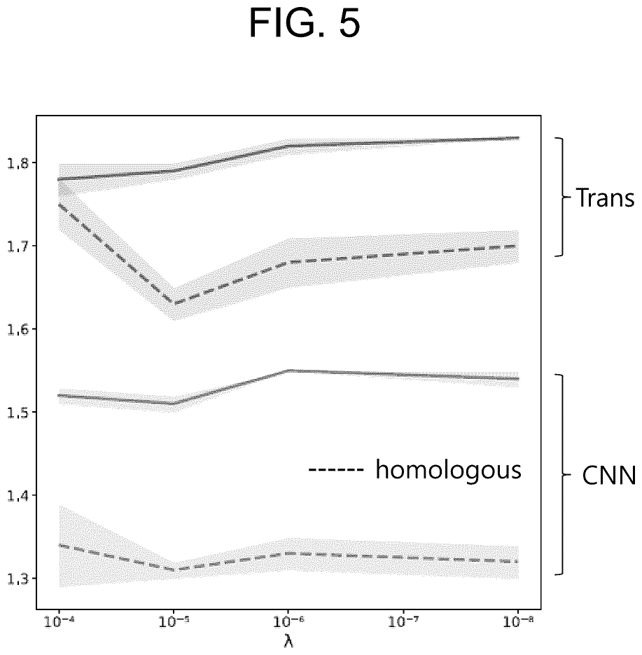

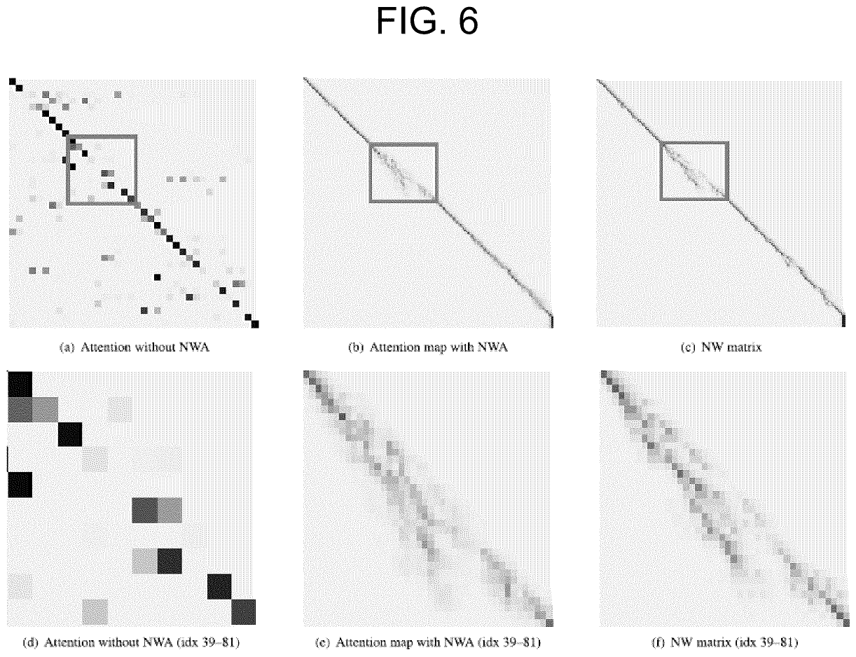

Hereinafter, experimental content of the DNA sequence embedding method according to the present invention will be described in more detail with reference to FIGS. 5 and 6.

Here, Qiita and DNA-Fountain were used as experimental datasets. Qiita provides human microbiome data, and DNA-Fountain provides data obtained from a DNA storage experiment. The DSEE model used a homologous sequence and a non-homologous sequence, and in this experiment, learning data was prepared in a manner similar to a NeuroSEED setting.

The Needleman-Wunsch Attention (NWA) framework 120 proposed in the present invention is a mechanism that transfers alignment information between DNA sequences in addition to a structure of an existing encoder, and can effectively learn the alignment information while maintaining the structure of the existing encoder. In the NWA framework, a parameter λ plays an important role in learning the alignment information and a distance structure. Here, λ is a parameter for adjusting a weight of the regularization term, and plays a role in balancing an NW loss and an encoder loss.

FIG. 5 is a diagram illustrating a result of scaling the regularization term weight adjustment parameter λ in a learning model of the NWA framework proposed in the present invention.

In FIG. 5, a result of scaling a λ value using a root mean square error (RMSE) in the Qiita dataset is shown. In the experiment, results according to the adjustment of the A value were analyzed when the embedding dimension was m=128. An optimal Δ value should be selected as a value working well for both the homologous sequence (homelogous pairs) and the non-homelogous sequence (non-homelogous pairs). In the experiment, the λ value was determined at a point where an exponent of the NW loss was adjusted to 1/10 of the encoder loss. This means that it is important to select an appropriate ratio so that the NW loss is not too greater or smaller than the encoder loss. The λ value may be flexibly adjusted depending on a dataset, and in the experiment, a default λ value is 10⁻⁵, but may be adjusted to a smaller value such as 10⁻⁶ or 10⁻⁸ when the performance is insufficient. It was shown that it is effective to set the λ value in a DNA-Fountain dataset to three times that used in the Qiita dataset.

Since a comprehensive evaluation of the proposed NWA framework 120 was performed using several encoders and performance changes were compared. The experiment was conducted at two embedding dimensions, that is, m=128 and m=256. The embedding dimension indicates a magnitude of a vector when a sequence is transformed into an embedding vector. In the experiment, a transformer and a CNN encoder structure were used. The transformer refers to a version of a NeuroSEED model that uses a transformer encoder, and the CNN refers to a version of the NeuroSEED model that uses a CNN encoder. Experimental results were reported as the RMSE, and a threshold K for identifying the homologous sequence was set to 40 according to a DSEE setting. The experimental results are shown in Table 1 below.

TABLE 1

| | | m = 128 | | m = 256 |
|---|---|---|---|---|
| Model | m = 128 | (ED ≤ K) | m = 256 | (ED ≤ K) |
| Transformer | 1.84 ± 0.02 | 1.73 ± 0.05 | 1.75 ± 0.05 | 1.64 ± 0.02 |
| Transformer + | 1.79 ± 0.01 | 1.63 ± 0.02 | 1.70 ± 0.02 | 1.62 ± 0.04 |
| NWA | | | | |
| CNN | 1.55 ± 0.01 | 1.29 ± 0.02 | 1.51 ± 0.01 | 1.32 ± 0.04 |
| CNN + NWA | 1.52 ± 0.01 | 1.29 ± 0.02 | 1.47 ± 0.01 | 1.27 ± 0.03 |
| DSEE | 3.92 ± 0.15 | 1.92 ± 0.05 | 3.99 ± 0.17 | 1.92 ± 0.06 |
| DSEE + NWA | 3.73 ± 0.05 | 1.85 ± 0.11 | 3.91 ± 0.15 | 1.89 ± 0.09 |

Table 1 above shows the RMSE and a K-homologous RMSE for each model. According to the results, the proposed NWA framework 120 improved embedding performance by accurately aligning sequence pairs and better reflecting edit distance information between the DNA sequences. In particular, it was shown that, in Qiita DNA data, the NWA framework 120 improved the embedding performance in both the homologous sequence and the non-homologous sequence, and the RMSE was reduced by up to 3% at the embedding dimension m=128 and up to 2% at m=256.

Evaluation results for the DNA-Fountain dataset are shown in Table 2 below.

TABLE 2

| Model | m = 128 | m = 256 |
|---|---|---|
| Trans | 2.01 ± 0.32 | 2.00 ± 0.33 |
| Trans + NWA | 1.74 ± 0.04 | 1.76 ± 0.06 |
| CNN | 1.61 ± 0.02 | 1.61 ± 0.01 |
| CNN + NWA | 1.61 ± 0.01 | 1.56 ± 0.04 |
| DSEE | 5.82 ± 0.07 | 6.51 ± 1.86 |
| DSEE + NWA | 5.54 ± 0.04 | 6.42 ± 1.29 |

According to the evaluation results in Table 2 above, the NWA framework 120 significantly improved the sequence embedding performance in the DNA-Fountain dataset. In particular, when the NWA framework 120 was applied to a transformer encoder, a performance improvement of up to 13% was achieved. Table 2 does not include RMSE results for homologous sequence pairs because the DNA-Fountain dataset used random sampling of the sequences.

FIG. 6 is a diagram illustrating a visualization of an original NW matrix and an attention map learned with NWA.

In FIG. 6, (a) shows an attention map learned without NWA, in which the alignment information between the sequences is not clear and portions with weak correlations appear sporadically. This shows that sequence alignment is not accurately reflected. (b) shows an attention map learned using the NWA framework, in which the alignment information is well reflected and a consistent pattern appears along a diagonal. This shows that the alignment relationship between the sequences is accurately reflected. (c) shows an NW matrix, in which optimal alignment between the sequences is shown, and information mainly aligned in a diagonal direction is clearly revealed. The attention map learned using the NWA in (b) shows a similar pattern to the original NW matrix shown in (c). (d) shows an enlargement of a square area in the attention map in (a), in which an attention map with sequence indices ranging from 39 to 81 is shown. It can be seen that a model learned without NWA still shows an irregular pattern with unclear alignment. (e) shows an enlargement of a square area in the attention map in (b), in which a regular and consistent alignment pattern along a diagonal is shown. (f) shows an enlargement of a square area (sequence indices 39 to 81) in the NW matrix in (c), in which sequence alignment information clearly appears along a diagonal and shows a consistent pattern, similar to the NWA attention map. In other words, it can be seen that the attention map generated by NWA has a similar structure to the original NW matrix. This shows that the proposed NWA framework provides reliable results for sequence alignment.

The DNA sequence embedding device and method based on an attention mechanism according to the present invention may be applied to an existing CNN or a transformer-based DNA sequence embedding network, and may be used to improve edit distance preserving performance in an actual DNA sequence dataset.

Although the preferred embodiments of the present invention have been described above, it will be understood by those skilled in the art that the present invention can be variously modified and changed without departing from the scope and spirit of the present invention described in the claims below.
[National Research Development Project Supporting the Present Invention]
[Project Serial No] 2710006677
[Project No] RS-2020-II201361
[Department] Ministry of Science and ICT
[Preject management (Professional) Institute] Institute of Information & Communications Technology Planning & Evaluation
[Research Project Name] Nurturing ICT and Broadcasting Innovation Talents
[Research Task Name] Artificial Intelligence Graduate School Support Project (Yonsei University)
[Project Performing Institute] University Industry Foundation, Yonsei University
[Research Period] 2024.01.01˜2024.12.31

### [Detailed Description of Main Elements]

100: DNA sequence embedding device based on attention mechanism
110: Encoder module
120: NWA framework
122: Decoder module
124: Cross-attention module
126: Matrix transformation module
128: Learning module
310: Multi-head self-attention (MSA) unit
320: Feedforward network (FFN) unit

What is claimed is:

1. A DNA sequence embedding device based on an attention mechanism comprising:
an encoder module configured to transform a DNA sequence into an embedding vector;
a decoder module configured to receive the embedding vector and transform the embedding vector into a sequence vector;
a cross-attention module configured to generate an attention map based on a correlation between the sequence vectors;
a matrix transformation module configured to transform the DNA sequence into a dynamic program-based matrix; and
a learning module configured to perform learning to minimize a loss between the attention map and the matrix.

2. The DNA sequence embedding device based on an attention mechanism of claim 1, wherein the encoder module represents the edit distance between the DNA sequences as a distance in a vector space to generate the embedding vector.

3. The DNA sequence embedding device based on an attention mechanism of claim 2, wherein the encoder module includes a neural network that is trained to minimize an edit distance loss function [symbol] _{enc} (θ) so that a difference between an actual edit distance between the DNA sequences and a distance between the embedding vectors is decreased.

4. The DNA sequence embedding device based on an attention mechanism of claim 1, wherein the decoder module transforms the embedding vectors so that information close to original sequences can be extracted, thereby allowing the sequence vectors to reflect alignment information between the original sequences.

5. The DNA sequence embedding device based on an attention mechanism of claim 4, wherein the decoder module includes
a multi-head self-attention (MSA) unit configured to perform first learning based on a plurality of attention heads to learn a correlation between a first element and a second element of the embedding vector; and
a feedforward network (FFN) unit configured to perform second learning based on a multilayer perceptron structure to learn a complex relationship through nonlinear transformation of a result of learning the correlation.

6. The DNA sequence embedding device based on an attention mechanism of claim 1, wherein the cross-attention module performs calculation of the correlation and a row-wise Softmax operation based on a query-key-value structure of the sequence vector to generate the attention map probabilistically representing a relationship between the sequence vectors.

7. The DNA sequence embedding device based on an attention mechanism of claim 1, wherein the matrix transformation module implements a dynamic program-based matrix as a Needleman-Wunsch (NW) matrix.

8. The DNA sequence embedding device based on an attention mechanism of claim 1, wherein the learning module measures a probabilistic difference between the attention map and the matrix and performs the learning through a regularization term for minimizing the difference.

9. A DNA sequence embedding method based on an attention mechanism performed in a DNA sequence embedding device based on an attention mechanism, the DNA sequence embedding method based on an attention mechanism comprising:
an encoder step of transforming a DNA sequence into an embedding vector;
a decoder step of receiving the embedding vector and transforming the embedding vector into a sequence vector;
a cross-attention step of generating an attention map based on a correlation between the sequence vectors;
a matrix transformation step of transforming the DNA sequence into a dynamic program-based matrix; and
a learning step of performing learning to minimize a loss between the attention map and the matrix.
